(19) United States
(12) Patent Application Publication
(10) Pub. No.: US 2010/0331960 A1
(43) Pub. Date: Dec. 30, 2010

(54) ENDOPROSTHESIS AND ENDOPROSTHESIS DELIVERY SYSTEM AND METHOD

(75) Inventors: Claude Clerc, Marlborough, MA (US); Martin Patrick Kilgannon, County Galway (IE)

Correspondence Address: Hoffmann & Baron, LLP, 6900 Jericho Turnpike, Syosset, NY, 11791, US

(73) Assignee: BOSTON SCIENTIFIC SCIMED, INC., Maple Grove, MN (US)

(21) Appl. No.: 12/821,277

(22) Filed: Jun. 23, 2010

Related U.S. Application Data
(60) Provisional application No. 61/221,590, filed on Jun. 30, 2009.

(51) Int. Cl. A61F 2/82

(52) U.S. Cl. 623/1.15

(57) ABSTRACT

An endoprosthesis includes a structure which is self-expandable from a reduced profile to an expanded profile. The structure has one or more longitudinal portions and a transverse central plane about which the one or more longitudinal portions are symmetric. A removable sheath retains the one or more longitudinal portions in the reduced profile. A release structure is coupled to the sheath for removal thereof from the one or more longitudinal portions to provide the self-expansion thereof to the expanded profile. A method for implanting the endoprosthesis into a body of a patient includes inserting the structure which is covered by the sheath into the body of the patient such that the structure has the reduced profile. The release structure is then actuated for removing the sheath from the one or more longitudinal portions to provide the self-expansion to the expanded profile.

### CROSS-REFERENCE TO RELATED APPLICATIONS

This application claims the benefit of U.S. Provisional Application No. 61/221,590, filed Jun. 30, 2009, the contents of which are incorporated herein by reference.

### FIELD OF THE INVENTION

The present invention relates generally to an endoprosthesis and, more specifically, to an endoprosthesis including an expandable medical structure covered by a removable sheath.

### BACKGROUND OF THE INVENTION

An endoprosthesis is implantable in the body of a patient, such as a blood vessel or other body cavity. The endoprosthesis includes a medical structure, such as a stent, which is compressible against restoring spring forces to a cross section which is reduced relative to an expanded cross section for the implantation. The medical structure may automatically expand to the expanded cross section for the implantation following removal of the restraining forces providing the compression.

The medical structure of the endoprosthesis may be compressed to the reduced cross section by being surrounded by a removable sheath which includes at least one thread. The thread extends away from the sheath when the sheath retains the medical structure in the radially compressed position. The thread is retractable from the sheath. Retraction of the thread from the sheath causes removal thereof from the medical structure resulting in the expansion thereof from the radially compressed position to the expanded cross section for implantation. The sheath may be defined by a meshwork produced by crocheting, knitting, tying, or other methods of mesh formation. The meshwork may be unraveled by retraction of the thread which removes the sheath from the compressed medical structure.

The expansion of the medical structure, which results from removal of the sheath, may generate forces which displace the medical structure longitudinally relative to the body cavity within which the endoprosthesis is located. Any forces, and the resultant displacement of the medical structure, are preferably limited.

### SUMMARY OF THE INVENTION

The endoprosthesis of the present invention includes a medical structure which is self-expandable from a reduced profile to an expanded profile. The reduced profile provides for inserting the medical structure into a body of a patient. The expanded profile provides for implanting the medical structure in the body of the patient. The medical structure has one or more longitudinal portions and a transverse central plane about which the one or more longitudinal portions are often symmetric. The present invention, however, is not so limited. For example, the medical structure may have a flare or varied diameter portion at one end or both ends, have tapered portions, have step portions, and/or the like. A removable sheath may cover the one or more longitudinal portions of the medical structure, and retains the one or more longitudinal portions in the reduced profile. The removable sheath may conform to the shape of the stent. A release structure is coupled to the sheath for removal thereof from the one or more longitudinal portions of the medical structure to provide the self-expansion of the one or more longitudinal portions to the expanded profile. A method for implanting the endoprosthesis into a body of a patient includes inserting the medical structure which is covered by the sheath into the body of the patient such that the medical structure has the reduced profile. Following the insertion, the release structure is actuated for removing the sheath from the one or more longitudinal portions of the medical structure to provide the self-expansion to the expanded profile.

The endoprosthesis provides for the removal of the sheath from selected longitudinal portions of the medical structure. The expansion of the selected portions may be coordinated to limit the forces which may be generated to longitudinally displace the medical structure relative to the body cavity within which the endoprosthesis is located. For example, the coordination of the selected longitudinal portions may provide for the medical structure to expand in a selected longitudinal direction which limits any longitudinal displacement of the medical structure relative to the body cavity.

These and other features of the invention will be more fully understood from the following description of specific embodiments of the invention taken together with the accompanying drawings.

### BRIEF DESCRIPTION OF THE DRAWINGS

In the drawings:

FIG. 1 is a schematic view of the endoprosthesis of the present invention, the endoprosthesis being shown as including a stent and a sheath, the stent being illustrated as having a reduced profile from being compressed by the sheath;

FIG. 2 is a schematic view of the endoprosthesis of FIG. 1, the sheath being shown as partially removed such that portions of the stent are expanded to an expanded profile;

FIG. 3 is a schematic view of the endoprosthesis of FIG. 1, the sheath being shown as partially removed further such that larger portions of the stent are expanded to the expanded profile;

FIG. 4 is a schematic view of the endoprosthesis of FIG. 1, the sheath being shown as completely removed such that the entire stent is expanded to the expanded profile;

FIG. 5 is a schematic view of the endoprosthesis of FIG. 1, showing the relative positions of the connections of the release structures to the sheath, and the directions of the removal of the sheath from the stent;

FIG. 6 is a schematic view of an alternative embodiment of the endoprosthesis of FIG. 1, showing the relative positions of the connections of the release structures to the sheath, and the directions of the removal of the sheath from the stent;

FIG. 7 is a schematic view of a further alternative embodiment of the endoprosthesis of FIG. 1, showing the relative positions of the connections of the release structures to the sheath, and the directions of the removal of the sheath from the stent;

FIG. 8 is a schematic view of a further alternative embodiment of the endoprosthesis of FIG. 1, showing the relative positions of the connections of the release structures to the sheath, and the directions of the removal of the sheath from the stent;

FIG. 9 is a schematic view of a further alternative embodiment of the endoprosthesis of FIG. 1, showing the relative positions of the connections of the release structures to the sheath, and the directions of the removal of the sheath from the stent; and

FIG. 10 is a schematic view of a further alternative embodiment of the endoprosthesis of FIG. 1, showing the relative positions of the connections of the release structures to the sheath, and the directions of the removal of the sheath from the stent.

Corresponding reference characters indicate corresponding parts throughout the several views of the drawings.

### DETAILED DESCRIPTION OF THE INVENTION

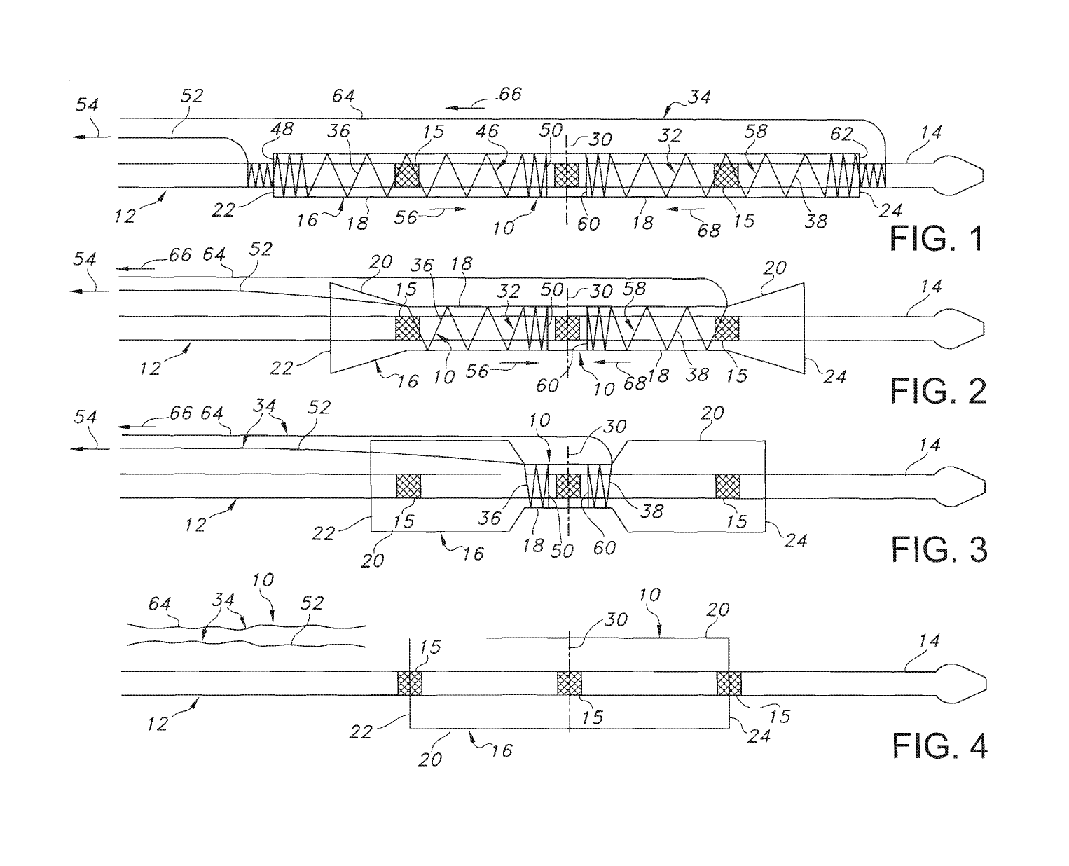

Referring to the drawings and more specifically to FIG. 1, the endoprosthesis 10 is used with a delivery system 12. The delivery system 12 includes an elongate inner structure 14 on which the endoprosthesis 10 is mounted. The delivery system 12 includes radiopaque markers 15 which are fixed to the elongate inner structure 14.

The endoprosthesis 10 includes a medical structure which, as shown in FIGS. 1 to 4, is an elongate tubular stent 16. Embodiments of the medical structure, other than the stent 16, are possible. The stent 16 is self-expandable from a reduced profile 18 to an expanded profile 20. Self-expandable stents include those that have a spring-like action which causes the stent to radially expand, or stents which expand due to the memory properties of the stent material for a particular configuration at a certain temperature. The stent 16 has proximal and distal ends 22, 24, and a transverse central plane 30 which intersects the stent midway between the proximal and distal ends 22, 24.

The stent 16 or stent filaments forming stent 16 may be formed of any suitable implantable material, including without limitation nitinol, stainless steel, cobalt-based alloy such as Elgiloy®, platinum, gold, titanium, titanium alloys, tantalum, niobium, polymeric materials and combinations thereof. Useful polymeric materials may include, for example, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, natural silk, polyvinyl chloride, polytetrafluoroethylene, including expanded polytetrafluoroethylene (ePTFE), fluorinated ethylene propylene copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, such as fully or partially halogenated polyethers, and copolymers and combinations thereof. Further, useful and nonlimiting examples of polymeric stent materials include poly(L-lactide) (PLLA), poly(D,L-lactide) (PLA), poly(glycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D,L-lactide-co-caprolactone) PLA/PCL), poly(glycolide-co-caprolactone) (PGA/PCL), polyphosphate ester) and the like. Wires made from polymeric materials may also include radiopaque materials, such as metallic-based powders, particulates or pastes which may be incorporated into the polymeric material. For example the radiopaque material may be blended with the polymer composition from which the polymeric wire is formed, and subsequently fashioned into the stent as described herein. Alternatively, the radiopaque material and/or radiopaque markers may be applied to the surface of the metal or polymer stent. In either embodiment, various radiopaque materials and their salts and derivatives may be used including, without limitation, bismuth, barium and its salts such as barium sulphate, tantulaum, tungsten, gold, platinum and titanium, to name a few. Additional useful radiopaque materials may be found in U.S. Pat. No. 6,626,936, which is herein incorporated in its entirely by reference. Metallic complexes useful as radiopaque materials are also contemplated. The stent may be selectively made radiopaque at desired areas along the wire or made be fully radiopaque, depending on the desired end-product and application. Further, the stent filaments may have an inner core of tantalum, gold, platinum, iridium or combination of thereof and an outer member or layer of nitinol to provide a composite wire for improved radiocapicity or visibility. Desirably, the inner core is platinum and the outer layer is nitinol. More desirably, the inner core of platinum represents about at least 10% of the wire based on the overall cross-sectional percentage. Moreover, nitinol that has not been treated for shape memory such as by heating, shaping and cooling the nitinol at its martensitic and austenitic phases, is also useful as the outer layer. Further details of such composite wires may be found in U.S. Patent Application Publication 2002/0035396 A1, the contents of which is incorporated herein by reference. Preferably, the stent filaments are made from nitinol, or a composite wire having a central core of platinum and an outer layer of nitinol.

The stent 16 may include one or more coiled stainless steel springs, helically wound coil springs including a heat-sensitive material, or expanding stainless steel stents formed of stainless steel wire in a zig-zag pattern. The stent 16 may be capable of radially expanding by radial or circumferential distension or deformation. The stent 16 may self-expand at one or more specific temperatures as a result of the memory properties of the material included in the stent for a specific configuration. Nitinol is a material which may be included in the stent 16 for providing radial expansion thereof by the memory properties of the nitinol based on one or more specific temperatures or the superelastic properties of nitinol.

The endoprosthesis 10 includes a tubular sheath 32 within which is located the stent 16 in coaxial relation therewith. The internal cross-sectional area of the sheath 32 is less than the outer cross-sectional area of the expanded profile 20 of the stent 16. Consequently, location of the stent 16 within the sheath 32 compresses the stent. The internal cross-sectional area of the sheath 32 is sized such that location of the stent 16 within the sheath compresses the stent to the reduced profile 18. The stent 16 is retained in the reduced profile 18 by the sheath 32. Any number of sheaths 32 may suitably be used. Further, the dimensions of sheaths 32 are also non-limiting. Further, if more than one sheath 32 is used, then the sheaths 32 may or may not overlap one and another. Further, if more than one sheath 32 is used, then the sequence of removal of the sheaths 32 may be in any suitable order, typically dependent upon the particular delivery procedure used by a practitioner.

The endoprosthesis 10 includes a release structure 34 which provides for removal of the sheath 32. Removal of the sheath 32 provides for self-expansion of the stent 16 to the expanded profile 20.

The stent 16, sheath 32, and release structure 34 are typically formed of monofilament or braided suture. The suture may be impregnated or coated with a lubricant such as polytetrafluoroethylene (PTFE) or silicone. The sheath 32, and release structure 34 may be formed of biocompatible materials, such as biocompatible polymers including those which are known. Such polymers may include fillers such as metals, carbon fibers, glass fibers or ceramics. Also, such polymers may include olefin polymers, polyethylene, polypropylene, polyvinyl chloride, polytetrafluoroethylene, expanded polytetrafluoroethylene, fluorinated ethylene propylene copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, such as fully or partially halogenated polyethers, copolymers, and combinations thereof. Also, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, and natural silk may be included in the stent 16, sheath 32, and release structure 34.

The stent 16, sheath 32, and release structure 34 may be treated with a therapeutic agent or agents. “Therapeutic agents”, “pharmaceuticals,” “pharmaceutically active agents”, “drugs” and other related terms may be used interchangeably herein and include genetic therapeutic agents, non-genetic therapeutic agents and cells. Therapeutic agents may be used singly or in combination. A wide variety of therapeutic agents can be employed in conjunction with the present invention including those used for the treatment of a wide variety of diseases and conditions (i.e., the prevention of a disease or condition, the reduction or elimination of symptoms associated with a disease or condition, or the substantial or complete elimination of a disease or condition).

Non-limiting examples of useful therapeutic agents include, but are not limited to, adrenergic agents, adrenocortical steroids, adrenocortical suppressants, alcohol deterrents, aldosterone antagonists, amino acids and proteins, ammonia detoxicants, anabolic agents, analeptic agents, analgesic agents, androgenic agents, anesthetic agents, anorectic compounds, anorexic agents, antagonists, anterior pituitary activators and suppressants, anthelmintic agents, anti-adrenergic agents, anti-allergic agents, anti-amebic agents, anti-androgen agents, anti-anemic agents, anti-anginal agents, anti-anxiety agents, anti-arthritic agents, anti-asthmatic agents, anti-atherosclerotic agents, antibacterial agents, anticholelithic agents, anticholelithogenic agents, anticholinergic agents, anticoagulants, anticoccidal agents, anticonvulsants, antidepressants, antidiabetic agents, antidiuretics, antidotes, antidyskinetics agents, anti-emetic agents, anti-epileptic agents, anti-estrogen agents, antifibrinolytic agents, antifungal agents, antiglaucoma agents, antihemophilic agents, antihemophilic Factor, antihemorrhagic agents, antihistaminic agents, antihyperlipidemic agents, antihyperlipoproteinemic agents, antihypertensives, antihypotensives, anti-infective agents, anti-inflammatory agents, antikeratinizing agents, antimicrobial agents, antimigraine agents, antimitotic agents, antimycotic agents, antineoplastic agents, anti-cancer supplementary potentiating agents, antineutropenic agents, antiobsessional agents, antiparasitic agents, antiparkinsonian drugs, antipneumocystic agents, antiproliferative agents, antiprostatic hypertrophydrugs, antiprotozoal agents, antipruritics, antipsoriatic agents, antipsychotics, antirheumatic agents, antischistosomal agents, antiseborrheic agents, antispasmodic agents, antithrombotic agents, antitussive agents, anti-ulcerative agents, anti-urolithic agents, antiviral agents, benign prostatic hyperplasia therapy agents, blood glucose regulators, bone resorption inhibitors, bronchodilators, carbonic anhydrase inhibitors, cardiac depressants, cardioprotectants, cardiotonic agents, cardiovascular agents, choleretic agents, cholinergic agents, cholinergic agonists, cholinesterase deactivators, coccidiostat agents, cognition adjuvants and cognition enhancers, depressants, diagnostic aids, diuretics, dopaminergic agents, ectoparasiticides, emetic agents, enzyme inhibitors, estrogens, fibrinolytic agents, free oxygen radical scavengers, gastrointestinal motility agents, glucocorticoids, gonad-stimulating principles, hemostatic agents, histamine H2 receptor antagonists, hormones, hypocholesterolemic agents, hypoglycemic agents, hypolipidemic agents, hypotensive agents, HMGCoA reductase inhibitors, immunizing agents, immunomodulators, immunoregulators, immunostimulants, immunosuppressants, impotence therapy adjuncts, keratolytic agents, LHRH agonists, luteolysin agents, mucolytics, mucosal protective agents, mydriatic agents, nasal decongestants, neuroleptic agents, neuromuscular blocking agents, neuroprotective agents, NMDA antagonists, non-hormonal sterol derivatives, oxytocic agents, plasminogen activators, platelet activating factor antagonists, platelet aggregation inhibitors, post-stroke and post-head trauma treatments, progestins, prostaglandins, prostate growth inhibitors, prothyrotropin agents, psychotropic agents, radioactive agents, repartitioning agents, scabicides, sclerosing agents, sedatives, sedative-hypnotic agents, selective adenosine A1 antagonists, adenosine A2 receptor antagonists (e.g., CGS 21680, regadenoson, UK 432097 or GW 328267), serotonin antagonists, serotonin inhibitors, serotonin receptor antagonists, steroids, stimulants, thyroid hormones, thyroid inhibitors, thyromimetic agents, tranquilizers, unstable angina agents, uricosuric agents, vasoconstrictors, vasodilators, vulnerary agents, wound healing agents, xanthine oxidase inhibitors, and the like, and combinations thereof.

Useful non-genetic therapeutic agents for use in connection with the present invention include, but are not limited to,
(a) anti-thrombotic agents such as heparin, heparin derivatives, urokinase, clopidogrel, and PPack (dextrophenylalanine proline arginine chloromethylketone);
(b) anti-inflammatory agents such as dexamethasone, prednisolone, corticosterone, budesonide, estrogen, sulfasalazine and mesalamine;
(c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors;
(d) anesthetic agents such as lidocaine, bupivacaine and ropivacaine;
(e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides;
(f) vascular cell growth promoters such as growth factors, transcriptional activators, and translational promotors;
(g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
(h) protein kinase and tyrosine kinase inhibitors (e.g., tyrphostins, genistein, quinoxalines);
(i) prostacyclin analogs;
(j) cholesterol-lowering agents;
(k) angiopoietins;
(l) antimicrobial agents such as triclosan, cephalosporins, aminoglycosides and nitrofurantoin;
(m) cytotoxic agents, cytostatic agents and cell proliferation affectors;
(n) vasodilating agents;
(o) agents that interfere with endogenous vasoactive mechanisms;
(p) inhibitors of leukocyte recruitment, such as monoclonal antibodies;
(q) cytokines;
(r) hormones;
(s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin;
(t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, diclomine);
(u) bARKct inhibitors;
(v) phospholamban inhibitors;
(w) Serca 2 gene/protein;
(x) immune response modifiers including aminoquizolines, for instance, imidazoquinolines such as resiquimod and imiquimod;
(y) human apolioproteins (e.g., AI, AII, AIII, AIV, AV, etc.);
(z) selective estrogen receptor modulators (SERMs) such as raloxifene, lasofoxifene, arzoxifene, miproxifene, ospemifene, PKS 3741, MF 101 and SR 16234;
(aa) PPAR agonists, including PPAR-alpha, gamma and delta agonists, such as rosiglitazone, pioglitazone, netoglitazone, fenofibrate, bexaotene, metaglidasen, rivoglitazone and tesaglitazar;
(bb) prostaglandin E agonists, including PGE2 agonists, such as alprostadil or ONO 8815Ly;
(cc) thrombin receptor activating peptide (TRAP);
(dd) vasopeptidase inhibitors including benazepril, fosinopril, lisinopril, quinapril, ramipril, imidapril, delapril, moexipril and spirapril;
(ee) thymosin beta 4;
(ff) phospholipids including phosphorylcholine, phosphatidylinositol and phosphatidylcholine; and
(gg) VLA-4 antagonists and VCAM-1 antagonists.
   The non-genetic therapeutic agents may be used individually or in combination, including in combination with any of the agents described herein.

Further examples of non-genetic therapeutic agents, not necessarily exclusive of those listed above, include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, zotarolimus, Epo D, dexamethasone, estradiol, halofuginone, cilostazole, geldanamycin, alagebrium chloride (ALT-711), ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well derivatives of the forgoing, among others.

Useful genetic therapeutic agents for use in connection with the present invention include, but are not limited to, anti-sense DNA and RNA as well as DNA coding for the various proteins (as well as the proteins themselves), such as (a) anti-sense RNA; (b) tRNA or rRNA to replace defective or deficient endogenous molecules; (c) angiogenic and other factors including growth factors such as acidic and basic fibroblast growth factors, vascular endothelial growth factor, endothelial mitogenic growth factors, epidermal growth factor, transforming growth factor α and β, platelet-derived endothelial growth factor, platelet-derived growth factor, tumor necrosis factor α, hepatocyte growth factor and insulin-like growth factor; (d) cell cycle inhibitors including CD inhibitors, and (e) thymidine kinase (“TK”) and other agents useful for interfering with cell proliferation. DNA encoding for the family of bone morphogenic proteins (“BMP's”) are also useful and include, but not limited to, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently desirably BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include, but not limited to, viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers such as polyvinylpyrrolidone (PVP), SP1017 (SUPRATEK), lipids such as cationic lipids, liposomes, lipoplexes, nanoparticles, or microparticles, with and without targeting sequences such as the protein transduction domain (PTD).

Cells for use in connection with the present invention may include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Numerous therapeutic agents, not necessarily exclusive of those listed above, have been identified as candidates for vascular treatment regimens, for example, as agents targeting restenosis (antirestenotics). Such agents are useful for the practice of the present invention and include one or more of the following:
(a) Ca-channel blockers including benzothiazapines such as diltiazem and clentiazem, dihydropyridines such as nifedipine, amlodipine and nicardapine, and phenylalkylamines such as verapamil;
(b) serotonin pathway modulators including: 5-HT antagonists such as ketanserin and naftidrofuryl, as well as 5-HT uptake inhibitors such as fluoxetine;
(c) cyclic nucleotide pathway agents including phosphodiesterase inhibitors such as cilostazole and dipyridamole, adenylate/Guanylate cyclase stimulants such as forskolin, as well as adenosine analogs;
(d) catecholamine modulators including α-antagonists such as prazosin and bunazosine, β-antagonists such as propranolol and α/β-antagonists such as labetalol and carvedilol;
(e) endothelin receptor antagonists such as bosentan, sitaxsentan sodium, atrasentan, endonentan;
(f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine;
(g) Angiotensin Converting Enzyme (ACE) inhibitors such as cilazapril, fosinopril and enalapril;
(h) ATII-receptor antagonists such as saralasin and losartin;
(i) platelet adhesion inhibitors such as albumin and polyethylene oxide;
(j) platelet aggregation inhibitors including cilostazole, aspirin and thienopyridine (ticlopidine, clopidogrel) and GP IIb/IIIa inhibitors such as abciximab, epitifibatide and tirofiban;
(k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C;
(l) cyclooxygenase pathway inhibitors such as aspirin, ibuprofen, flurbiprofen, indomethacin and sulfinpyrazone;
(m) natural and synthetic corticosteroids such as dexamethasone, prednisolone, methprednisolone and hydrocortisone;
(n) lipoxygenase pathway inhibitors such as nordihydroguairetic acid and caffeic acid;
(o) leukotriene receptor antagonists; (p) antagonists of E- and P-selectins;
(q) inhibitors of VCAM-1 and ICAM-1 interactions;
(r) prostaglandins and analogs thereof including prostaglandins such as PGE1 and PGI2 and prostacyclin analogs such as ciprostene, epoprostenol, carbacyclin, iloprost and beraprost;
(s) macrophage activation preventers including bisphosphonates;
(t) HMG-CoA reductase inhibitors such as lovastatin, pravastatin, atorvastatin, fluvastatin, simvastatin and cerivastatin;
(u) fish oils and omega-3-fatty acids;
(v) free-radical scavengers/antioxidants such as probucol, vitamins C and E, ebselen, trans-retinoic acid, SOD (orgotein) and SOD mimics, verteporfin, rostaporfin, AGI 1067, and M 40419;
(w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives;
(x) matrix metalloprotease (MMP) pathway inhibitors such as marimastat, ilomastat, metastat, batimastat, pentosan polysulfate, rebimastat, incyclinide, apratastat, PG 116800, RO 1130830 or ABT 518;
(y) cell motility inhibitors such as cytochalasin B;
(z) antiproliferative/antineoplastic agents including antimetabolites such as purine antagonists/analogs (e.g., 6-mercaptopurine and pro-drugs of 6-mercaptopurine such as azathioprine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), olimus family drugs (e.g., sirolimus, everolimus, tacrolimus, zotarolimus, etc.), cerivastatin, flavopiridol and suramin;
(aa) matrix deposition/organization pathway inhibitors such as halofuginone or other quinazolinone derivatives, pirfenidone and tranilast;
(bb) endothelialization facilitators such as VEGF and RGD peptide;
(cc) blood rheology modulators such as pentoxifylline and
(dd) glucose cross-link breakers such as alagebrium chloride (ALT-711).
   These therapeutic agents may be used individually or in combination, including in combination with any of the agents described herein.

Numerous additional therapeutic agents useful for the practice of the present invention are also disclosed in U.S. Pat. No. 5,733,925 to Kunz, the contents of which is incorporated herein by reference.

A wide range of therapeutic agent loadings may used in connection with the dosage forms of the present invention, with the pharmaceutically effective amount being readily determined by those of ordinary skill in the art and ultimately depending, for example, upon the condition to be treated, the nature of the therapeutic agent itself, the tissue into which the dosage form is introduced, and so forth.

The delivery system 12 provides for location of the stent 16 within the body of the patient by mounting the stent 10 on the inner structure 14 in coaxial relation therewith. The stent 16 is longitudinally positioned relative to the inner structure 14 such that the longitudinal center of the stent has generally the same axial position as one of the radiopaque markers 15. The sheath 32 is placed over the stent 16 for compression thereof to the reduced profile 18. The delivery system 12 and endoprosthesis 10 mounted thereon is moved to the desired location within the body of the patient. The positioning of the stent 16 is facilitated by the radiopaque markers 15, such as the radiopaque marker which has generally the same axial position as the longitudinal center of the stent. An advantageous application of the stent 16 is for the treatment of a stricture within a vessel in the body of the patient. The treatment may be provided by employing the delivery system 12 to locating the endoprosthesis 10 within the vessel or lumen such the stent 16 is located at the stricture. The stent 16 may be more beneficially located by positioning the radiopaque marker 15, which has generally the same axial position as the longitudinal center of the stent, at generally the same axial position as the longitudinal center of the stricture.

The sheath 32 is defined by one or more sutures 36 woven into one or more sections of crocheted material 38 which retains the stent 16 in the reduced profile 18, as shown in FIGS. 1 to 3. As used herein, sutures 36 may be described as filaments 36, including monofilaments and/or multifilaments. Any suitable suture or filament material may be used with the present invention. The cross-sectional configuration of the suture or filament material may also include any suitable configuration. Embodiments of the crocheted material 38 are disclosed in U.S. Pat. Nos. 5,653,748 and 6,019,785, which are hereby incorporated by reference herein. The crocheted material 38 is woven such that one or more end portions of the sutures 36, which define corresponding release structures 34, extend away from the crocheted material. The weaving of the sutures 36 into the crocheted material 38 provides for displacement of the one or more release structures 34 away from the crocheted material to cause unraveling thereof. Alternative embodiments of the release structure 34 are possible which cause unraveling or other removal of the woven material or other structure of the sheath 32 from the stent 16. Other embodiments of the sheath 32 are possible, including but limited to, other patterns and structures including braids, weaves, twists and/or knots.

The sheath 32 includes a proximal section 46 of the crocheted material 38 which surrounds the stent 16 in coaxial relation therewith such the proximal section is located between the proximal end 22 and transverse central plane 30, as shown in FIG. 1. The proximal section 46 has proximal and distal ends 48, 50. The release structure 34 includes a proximal release structure 52 defined by a suture 36 which is woven into the proximal section 46 and extends from the proximal end 48. The proximal release structure 52 is defined by a portion of a single suture 36 the weaving of which forms the proximal section 46. The weaving of the proximal release structure 52 into the proximal section 46 provides for displacement of the proximal release structure in a direction 54 which is away from the proximal section to cause unraveling thereof. The unraveling of the proximal section 46 commences from the proximal end 48 and proceeds in an axial direction 56 relative to the sheath 32 toward the distal end 50. However, any unraveling scheme is contemplated with various stents and/or steps, points or aspects of delivery.

The sheath 32 includes a distal section 58 of the crocheted material 38 which surrounds the stent 16 in coaxial relation therewith such the distal section is located between the distal end 24 and transverse central plane 30, as shown in FIG. 1. The distal section 58 has proximal and distal ends 60, 62. The release structure 34 includes a distal release structure 64 defined by a suture 36 which is woven into the distal section 58 and extends from the distal end 62. The distal release structure 64 is defined by a portion of a single suture 36 the weaving of which forms the distal section 58. The weaving of the distal release structure 64 into the distal section 58 provides for displacement of the distal release structure in a direction 66 which is away from the distal section to cause unraveling thereof. The unraveling of the distal section 58 commences from the distal end 62 and proceeds in an axial direction 68 relative to the sheath 32 toward the proximal end 60. However, any unraveling scheme is contemplated with various stents and/or steps, points or aspects of delivery.

The distal section 58 has axial and transverse dimensions which are generally the same as the axial and transverse dimensions of the proximal section 46. The axial locations of the proximal and distal sections 46, 58 are symmetrical relative to the transverse central plane 30. The proximal and distal sections 46, 58 are located relative to the stent 16 such that the distal and proximal ends 50, 60 are separated axially from the transverse central plane 30. Alternative embodiments of the sheath 32 are possible in which the distal and proximal ends 50, 60 extend to the transverse central plane 30 such that the distal and proximal ends 50, 60 contact one another.

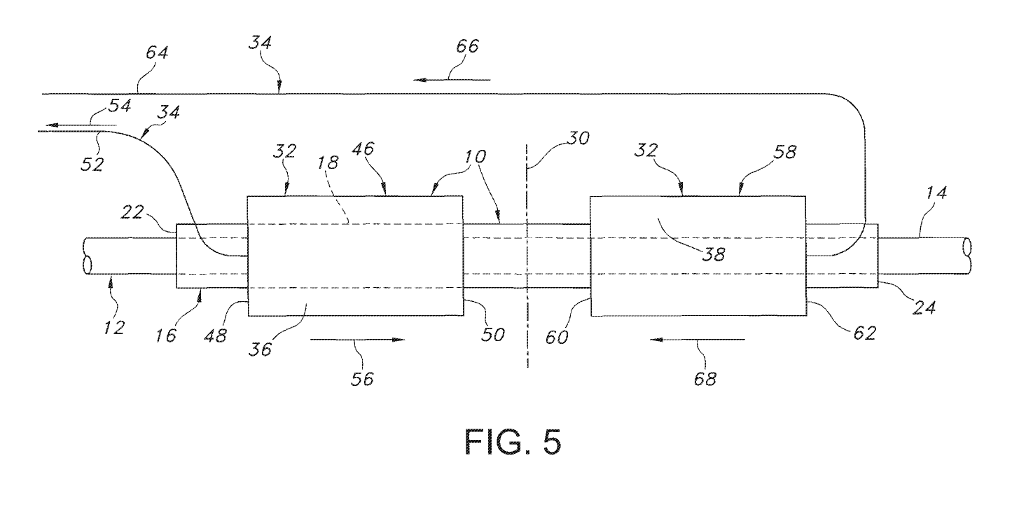

The corresponding dimensions of the proximal and distal sections 46, 58, the axial locations thereof relative to the transverse central plane 30, and the locations of the proximal and distal release structures 52, 64 at the proximal and distal ends 48, 62 provide for unraveling of the proximal and distal sections at generally the same rates in the axial directions 56, 68 toward the transverse central plane when the proximal and distal release structures are simultaneously displaced in the directions 54, 66 away from the proximal and distal sections. As shown in FIG. 5, the portions of the proximal and distal release structures 52, 64 which are remote from the sheath 32 are displaced proximally relative thereto in the directions 54, 66.

The inner structure 14 has an interior longitudinal cavity through which the proximal and distal release structures 52, 64 extend in the proximal direction. The inner structure 14 has one or more ports through which the proximal and distal release structures 52, 64 may extend from the proximal and distal sections 46, 58 into the cavity. The present invention, however, is not so limited. For example, the proximal and distal release structures may be external to the delivery device. The inner structure 14 has one or more grooves or channels formed on the inner surface thereof. The proximal and distal release structures 52, 64 are located in the grooves or channels and translate therein through the cavity in the inner structure 14. The proximal and distal release structures 52, 64 may be located in separate, respective channels or grooves, or may be commonly located in a single channel or groove. The proximal and distal release structures 52, 64 are available for manipulation by the user at a location on the inner structure 14 which is sufficiently remote from the proximal and distal sections 46, 58.

In alternative embodiments of the proximal and distal release structures 52, 64, the portions thereof which are remote from the sheath 32 are displaced in distal or other directions relative to the sheath 32.

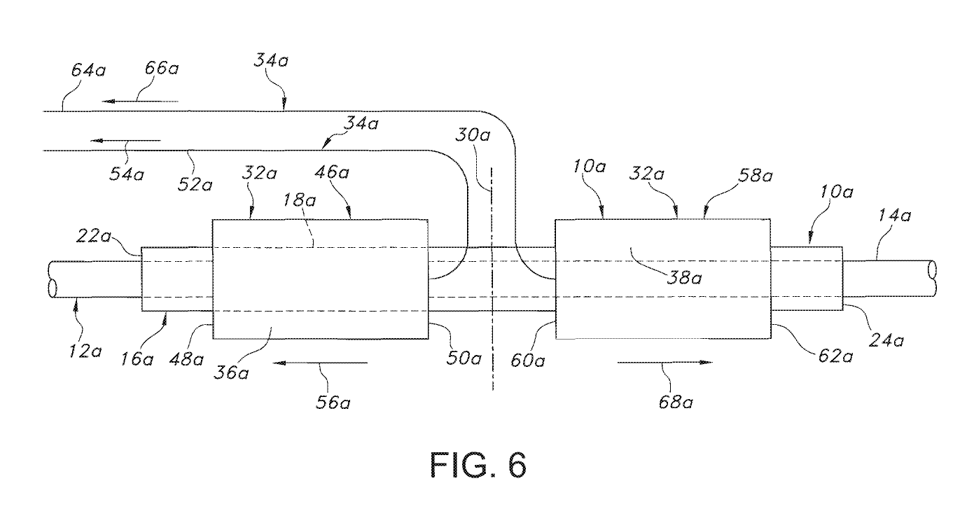

An alternative embodiment of the endoprosthesis 10a is shown in FIG. 6. Parts illustrated in FIG. 6 which correspond to parts illustrated in FIGS. 1 to 5 have, in FIG. 6, the same reference numeral as in FIGS. 1 to 5 with the addition of the suffix “a”. In this alternative embodiment, the proximal section 46a is woven such that the proximal release structure 52a extends from the distal end 50a. The proximal release structure 52a is defined by a portion of a single suture 36a the weaving of which forms the proximal section 46a. The weaving of the proximal release structure 52a into the proximal section 46a can provide for displacement of the proximal release structure in a direction 54a which is away from the proximal section to cause unraveling thereof. The unraveling of the proximal section 46a may commence from the distal end 50a and proceeds in an axial direction 56a relative to the sheath 32a toward the proximal end 48a.

The distal section 58a is woven such that the distal release structure 64a extends from the proximal end 60a. The distal release structure 64a is defined by a portion of a single suture 36a the weaving of which forms the distal section 58a. The present invention, however, is not so limited. For example, multiple filaments may be utilized as the suture 36a. The weaving of the distal release structure 64a into the distal section 58a provides for displacement of the distal release structure in a direction 66a which is away from the distal section to cause unraveling thereof. The unraveling of the distal section 58a commences from the proximal end 60a and proceeds in an axial direction 68a relative to the sheath 43a toward the distal end 62a.

The corresponding dimensions of the proximal and distal sections 46a, 58a, the axial locations thereof relative to the transverse central plane 30a, and the locations of the proximal and distal release structures 52a, 64a at the distal and proximal ends 50a, 60a can provide for unraveling of the proximal and distal sections at generally the same rates in the axial directions 56a, 68a away from the transverse central plane when the proximal and distal release structures are simultaneously displaced in the directions Ma, 66a away from the proximal and distal sections.

As shown in FIG. 6, the portions of the proximal and distal release structures 52a, 64a which are remote from the sheath 32a are displaced proximally relative to the sheath in the directions Ma, 66a. Alternative embodiments may provide for displacement of the proximal and distal release structures 52a, 64a distally relative to the sheath 32a, or in other directions.

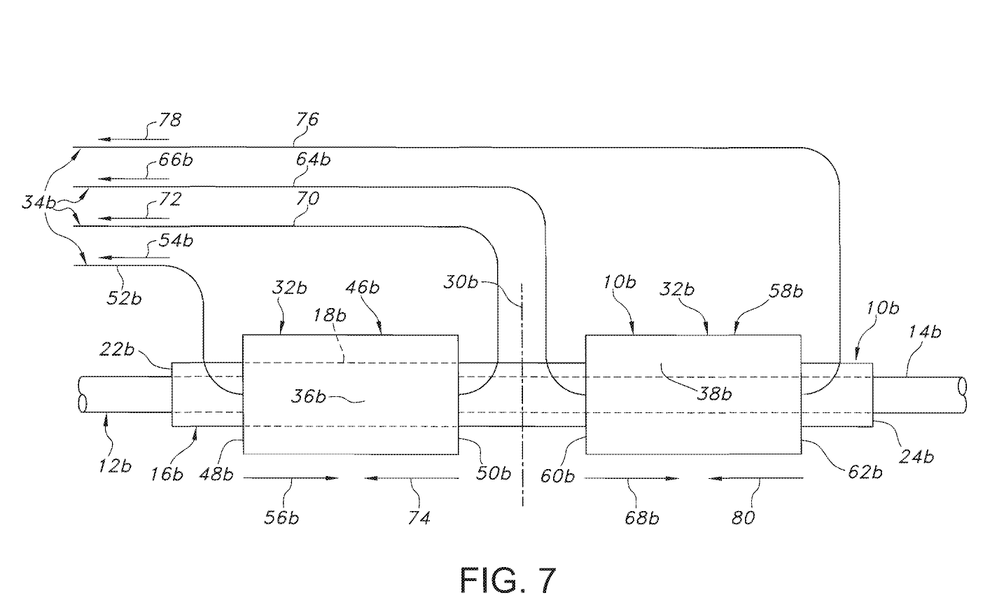

An alternative embodiment of the endoprosthesis 10b is shown in FIG. 7. Parts illustrated in FIG. 7 which correspond to parts illustrated in FIGS. 1 to 5 have, in FIG. 7, the same reference numeral as in FIGS. 1 to 5 with the addition of the suffix “b”. In this alternative embodiment, the proximal section 46b is woven such that the proximal release structure 52b extends from the proximal end 48b and a supplemental proximal release structure 70 extends from the distal end 50b. The proximal and supplemental proximal release structures 52b, 70 are defined by portions of a single suture 36b the weaving of which forms the proximal section 46b. The weaving of the proximal and supplemental proximal release structures 52b, 70 into the proximal section 46b can provide for displacement of the release structures in the directions 54b, 72 which are away from the proximal section to cause unraveling thereof. The unraveling of the proximal section 46b which results from the displacement of the proximal release structure 52b may commence from the proximal end 48b and proceeds in an axial direction 56b relative to the sheath 32b toward the transverse central plane 30b. The unraveling of the proximal section 46b which results from the displacement of the supplemental proximal release structure 70 may commence from the distal end 50b and proceeds in an axial direction 74 relative to the sheath 32b away from the transverse central plane 30b. The weaving of the proximal section 46b can provide for the respective unravelings thereof which result from the displacements of the proximal and supplemental proximal release structures 52b, 70 to meet at an axial location between the proximal and distal ends 48b, 50b to complete the unraveling of the proximal section.

The distal section 58b is woven such that the distal release structure 64b extends from the proximal end 60b and a supplemental distal release structure 76 extends from the distal end 62b. The distal and supplemental distal release structures 64b, 76 are defined by portions of a single suture 36b the weaving of which forms the distal section 58b. The weaving of the distal and supplemental distal release structures 64b, 76 into the distal section 58b can provide for displacement of the release structures in directions 66b, 78 which are away from the distal section to cause unraveling thereof. The unraveling of the distal section 58b which results from the displacement of the distal release structure 64b may commence from the proximal end 60b and proceeds in an axial direction 68b relative to the sheath 32b away from the transverse central plane 30b. The unraveling of the distal section 58b which results from the displacement of the supplemental distal release structure 76 may commence from the distal end 62b and proceeds in an axial direction 80 relative to the sheath 32b toward the transverse central plane 30b. The weaving of the distal section 58b can provide for the respective unravelings thereof which result from the displacements of the distal and supplemental distal release structures 64b, 76 to meet at an axial location between the proximal and distal ends 60b, 62b to complete the unraveling of the distal section.

The corresponding dimensions of the proximal and distal sections 46b, 58b, the axial locations thereof relative to the transverse central plane 30b, the locations of the proximal and supplemental proximal release structures 52b, 70 at the proximal and distal ends 48a, 50a, and the locations of the distal and supplemental distal release structures 64b, 76 at the proximal and distal ends 60b, 62b, provide for unraveling of the proximal and distal sections at generally the same rates in the axial directions 56b, 74, 68b, 80 toward the respective axial locations between the proximal and distal ends 48b, 50b, and proximal and distal ends 60b, 62b when the proximal and distal release structures are simultaneously displaced in the directions Mb, 72, 66b, 78 away from the proximal and distal sections.

As shown in FIG. 7, the portions of the proximal and supplemental proximal release structures 52b, 70, and distal and supplemental distal release structures 64b, 76 which are remote from the sheath 32b are displaced proximally relative thereto in the directions Mb, 72, 66b, 78. Alternative embodiments may provide for displacement of the proximal and supplemental proximal release structures 52b, 70, and distal and supplemental distal release structures 64b, 76 distally relative to the sheath 32b, or in other directions.

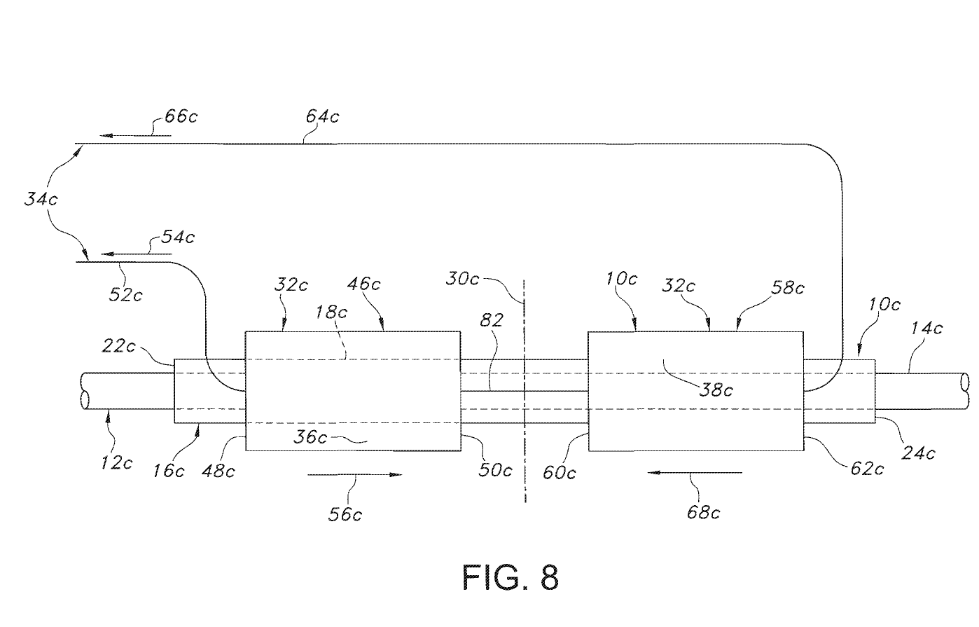

An alternative embodiment of the endoprosthesis 10c is shown in FIG. 8. Parts illustrated in FIG. 8 which correspond to parts illustrated in FIGS. 1 to 5 have, in FIG. 8, the same reference numeral as in FIGS. 1 to 5 with the addition of the suffix “c”. In this alternative embodiment, the proximal section 46c is woven such that the proximal release structure 52c extends from the proximal end 48c and an intermediate structure 82 extends from the distal end 50c. The proximal release structure 52c and intermediate structure 82 may be defined by portions of suture 36c the weaving of which forms the proximal section 46c. The weaving of the proximal release structure 52c and intermediate structure 82 into the proximal section 46c can provide for displacement of the proximal release structure in a direction 54c which is away from the proximal section to cause unraveling thereof. The unraveling of the proximal section 46c may commence from the proximal end 48c and proceeds in an axial direction 56c relative to the sheath 32c toward the distal end 50c.

The distal section 58c is woven such that the distal release structure 64c extends from the distal end 62c and the intermediate structure 82 extends from the proximal end 60c. The distal release structure 64c and intermediate structure 82 may be defined by portions of a single suture 36c the weaving of which forms the distal section 58c. Consequently, the intermediate structure 82, proximal and distal sections 46c, 58c, and proximal and distal release structures 52c, 64c, may be defined by portions of a single suture 36c.

The weaving of the distal release structure 64c and intermediate structure 82 into the distal section 58c can provide for displacement of the distal release structure in a direction 66c which is away from the distal section to cause unraveling thereof. The unraveling of the distal section 58c may commence from the distal end 62c and proceeds in an axial direction 68c relative to the sheath 32c toward the proximal end 60c.

The corresponding dimensions of the proximal and distal sections 46c, 58c, the axial locations thereof relative to the transverse central plane 30c, and the locations of the proximal and distal release structures 52c, 64c at the proximal and distal ends 48c, 62c can provide for unraveling of the proximal and distal sections at generally the same rates in the axial directions 56c, 68c toward the transverse central plane when the proximal and distal release structures are simultaneously displaced in the directions 54c, 66c away from the proximal and distal sections. Upon completion of the unraveling of the proximal and distal sections 46c, 58c, the proximal and distal release structures 52c, 64c coincide with the intermediate structure 82.

As shown in FIG. 8, the portions of the proximal and distal release structures 52c, 64c which are remote from the sheath 32c can be displaced proximally relative thereto in the directions 54c, 66c. Alternative embodiments may provide for displacement of the proximal and distal release structures 52c, 64c distally relative to the sheath 32c, or in other directions.

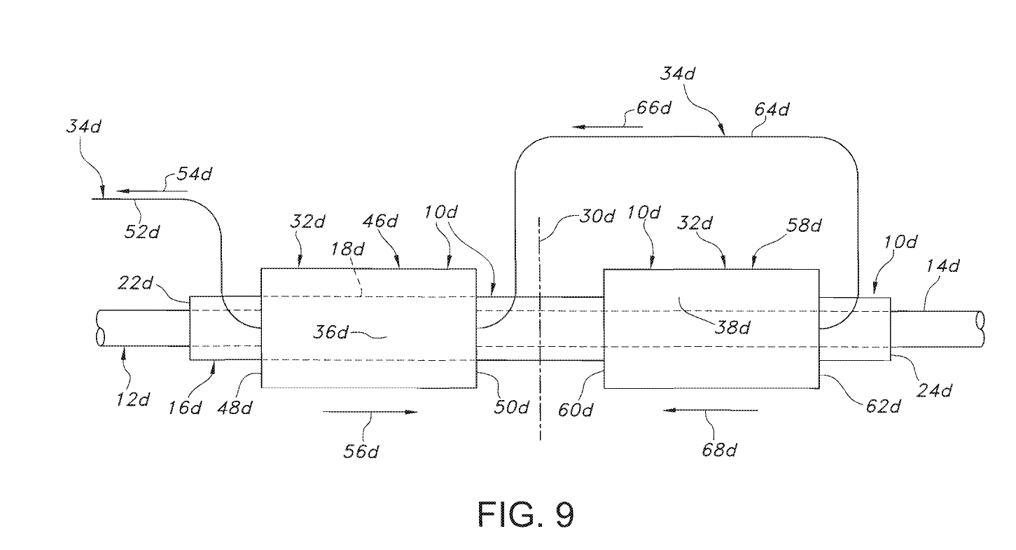

An alternative embodiment of the endoprosthesis 10d is shown in FIG. 9. Parts illustrated in FIG. 9 which correspond to parts illustrated in FIGS. 1 to 5 have, in FIG. 9, the same reference numeral as in FIGS. 1 to 5 with the addition of the suffix “d”. In this alternative embodiment, the proximal section 46d is woven such that the proximal release structure 52d extends from the proximal end 48d and the distal release structure 64d extends from the distal end 50d. The proximal and distal release structures 52d, 64d may be defined by portions of a single suture 36d the weaving of which forms the proximal section 46d. The weaving of the proximal release structure 52d into the proximal section 46d can provide for displacement of the proximal release structure in a direction 54d which is away from the proximal section to cause unraveling thereof. The unraveling of the proximal section 46d may commence from the proximal end 48d and proceeds in an axial direction 56d relative to the sheath 32d toward the distal end 50d.

The distal section 58d is woven such that the distal release structure 64d extends from the distal end 62d. The distal release structure 64d may be defined by a portion of a single suture 36d the weaving of which forms the distal section 58d. The proximal and distal sections 46d, 58d, and proximal and distal release structures 52d, 64d may be defined by portions of a single suture 36d. The weaving of the distal release structure 64d into the distal section 58d can provide for displacement of the distal release structure in a direction 66d which is away from the distal section to cause unraveling thereof. The unraveling of the distal section 58d may commence from the distal end 62d and proceeds in an axial direction 68d relative to the sheath 32d toward the proximal end 60d.

The extension of the distal release structure 64d from the distal ends 50d, 62d, and the locations of the proximal release structure 52d and distal release structure at the proximal and distal ends 48d, 62d can provide for the unraveling of the proximal section 46d to be followed by the unraveling of the distal section 58d. More specifically, the proximal release structure 52d can be displaced away from the sheath 32d in the direction 54d which results in the unraveling of the proximal section 46d in the axial direction 56d toward the transverse central plane 30d. Upon completion of the unraveling of the proximal section 46d, the proximal release structure 52d coincides with the distal release structure 64d. Continued displacement of the proximal and distal release structures 52d, 64d away from the sheath 32d in the directions 54d, 66d results in the unraveling of the distal section 58d in the axial direction 68d toward the transverse central plane 30d.

As shown in FIG. 9, the portion of the proximal release structure 52d which is remote from the sheath 32d can be displaced proximally relative thereto in the direction 54d. Alternative embodiments may provide for displacement of the proximal and distal release structures 52d, 64d distally relative to the sheath 32d, or in other directions.

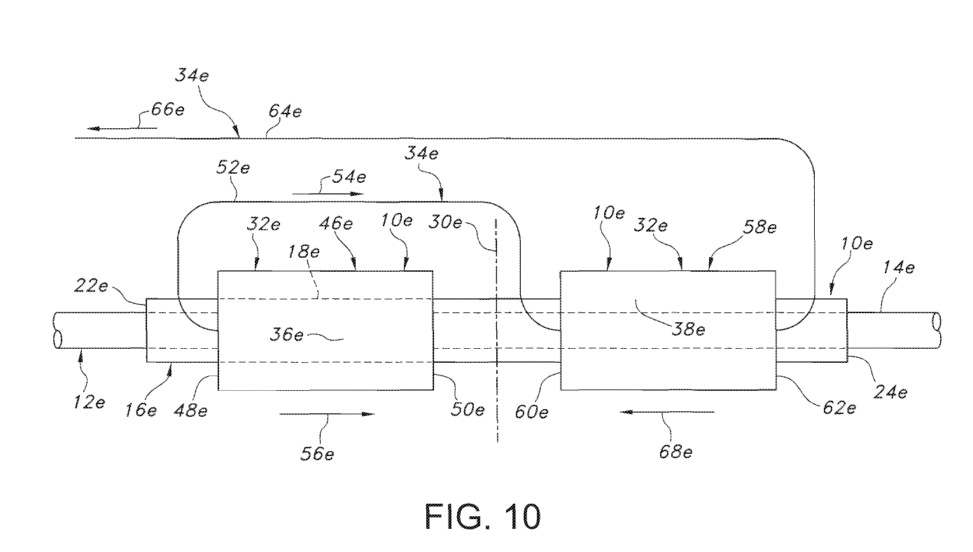

An alternative embodiment of the endoprosthesis 10e is shown in FIG. 10. Parts illustrated in FIG. 10 which correspond to parts illustrated in FIGS. 1 to 5 have, in FIG. 10, the same reference numeral as in FIGS. 1 to 5 with the addition of the suffix “e”. In this alternative embodiment, the proximal section 46e is woven such that the proximal release structure 52e extends from the proximal end 48e. The proximal release structure 52e may be defined by a portion of a single suture 36e the weaving of which forms the proximal section 46e. The weaving of the proximal release structure 52e into the proximal section 46e can provide for displacement of the proximal release structure in a direction 54e which is away from the proximal section to cause unraveling thereof. The unraveling of the proximal section 46e may commence from the proximal end 48e and proceeds in an axial direction 56e relative to the sheath 32e toward the distal end 50e.

The distal section 58e is woven such that the proximal release structure 52e extends from the proximal end 60e, and the distal release structure 64e extends from the distal end 62e. The proximal and distal release structures 52e, 64e may be defined by portions of a single suture 36e the weaving of which forms the distal section 58e. Consequently, the proximal and distal sections 46e, 58e, and proximal and distal release structures 52e, 64e may be defined by portions of a single suture 36e. The weaving of the distal release structure 64e into the distal section 58e can provide for displacement of the distal release structure in a direction 66e which is away from the distal section to cause unraveling thereof. The unraveling of the distal section 58e may commence from the distal end 62e and proceeds in an axial direction 68e relative to the sheath 32e toward the proximal end 60e.

The extension of the proximal release structure 52e from the proximal ends 48e, 60e, and the locations of the proximal release structure and distal release structure 64e at the proximal and distal ends 48e, 62e can provide for the unraveling of the distal section 58e to be followed by the unraveling of the proximal section 46e. More specifically, the distal release structure 64e can be displaced away from the sheath 32e in the direction 66e which results in the unraveling of the distal section 58e in the axial direction 68e toward the transverse central plane 30e. Upon completion of the unraveling of the distal section 58e, the distal release structure 64e coincides with the proximal release structure 52e. Continued displacement of the proximal and distal release structures 52e, 64e away from the sheath 32e in the directions 54e, 66e results in the unraveling of the proximal section 46e in the axial direction 56e toward the transverse central plane 30e. Directions 54e and 66e may be opposite directions when the distal section 58e is finished being released and the suture may be pulled along direction 66e. As shown in FIG. 10, the portion of the distal release structure 64e which is remote from the sheath 32e is displaced proximally relative thereto in the direction 66e. Alternative embodiments may provide for displacement of the distal release structure 64e distally relative to the sheath 32e, or in other directions.

Various stent types and stent constructions may be employed in the invention. Among the various stents useful include, without limitation, self-expanding stents and balloon expandable extents. The stents may be capable of radially contracting, as well and in this sense can best be described as radially distensible or deformable. Self-expanding stents include those that have a spring-like action which causes the stent to radially expand, or stents which expand due to the memory properties of the stent material for a particular configuration at a certain temperature. Nitinol is one material which has the ability to perform well while both in spring-like mode, as well as in a memory mode based on temperature. Other materials are of course contemplated, such as stainless steel, platinum, gold, titanium and other biocompatible metals, as well as polymeric stents. The configuration of the stent may also be chosen from a host of geometries. For example, wire stents can be fastened into a continuous helical pattern, with or without a wave-like or zig-zag in the wire, to form a radially deformable stent. Individual rings or circular members can be linked together such as by struts, sutures, welding or interlacing or locking of the rings to form a tubular stent. Tubular stents useful in the present invention also include those formed by etching or cutting a pattern from a tube. Such stents are often referred to as slotted stents. Furthermore, stents may be formed by etching a pattern into a material or mold and depositing stent material in the pattern, such as by chemical vapor deposition or the like. Examples of various stent configurations are shown in U.S. Pat. Nos. 4,503,569 to Dotter; 4,733,665 to Palmaz; 4,856,561 to Hillstead; 4,580,568 to Gianturco; 4,732,152 to Wallsten, 4,886,062 to Wiktor, and 5,876,448 to Thompson, all of whose contents are incorporated herein by reference.

With any embodiment, the endoprosthesis 10 may be used for a number of purposes including to maintain patency of a body lumen, vessel or conduit, such as in the coronary or peripheral vasculature, esophagus, trachea, bronchi colon, biliary tract, urinary tract, prostate, brain, and the like. The devices of the present invention may also be used to support a weakened body lumen or to provide a fluid-tight conduit for a body lumen.

While the invention has been described by reference to certain preferred embodiments, it should be understood that numerous changes could be made within the spirit and scope of the inventive concept described. Accordingly, it is intended that the invention not be limited to the disclosed embodiments, but that it have the full scope permitted by the language of the following claims.

1-17. (canceled)

18. An endoprosthesis comprising:
a structure which is self-expandable from a reduced profile to an expanded profile, said reduced profile providing for inserting said structure into a body of a patient, said expanded profile providing for implanting said structure in the body of the patient, said structure having one or more longitudinal portions and a transverse central plane;
a removable sheath covering said one or more longitudinal portions of said structure, said sheath retaining said one or more longitudinal portions in said reduced profile; and
a release structure coupled to said sheath for removing said sheath from said one or more longitudinal portions of said structure to provide said self-expansion of said one or more longitudinal portions to said expanded profile.

19. The endoprosthesis according to claim 18, wherein said one or more longitudinal portions are symmetric.

20. The endoprosthesis according to claim 18, wherein said one or more longitudinal portions are non-symmetric.

21. The endoprosthesis according to claim 18, wherein said sheath comprises a filament which is woven into a crocheted material,
said release structure comprising a portion of said filament which extends from said sheath such that displacement of said release structure relative to said sheath causes unraveling thereof for said removal of said sheath.

22. The endoprosthesis according to claim 18, wherein said structure comprises a stent.

23. The endoprosthesis according to claim 18, wherein said sheath comprises a distal section and a proximal section located such that said transverse central plane is between said distal and proximal sections, said distal and proximal sections being symmetric about said transverse central plane such that said distal and proximal sections cover corresponding ones of said longitudinal portions.

24. The endoprosthesis according to claim 18, wherein said sheath comprises a distal section and a proximal section located such that said transverse central plane is between said distal and proximal sections, said distal and proximal sections being disposed about said transverse central plane such that said distal and proximal sections cover corresponding ones of said longitudinal portions.

25. The endoprosthesis according to claim 18, wherein said distal and proximal sections comprise a crocheted material which is unraveled for said removal of said sheath.

26. The endoprosthesis according to claim 25, wherein said crocheted material of said distal and proximal sections is woven such that said proximal and distal sections unravel in opposite longitudinal directions relative to one and another.

27. The endoprosthesis according to claim 25, wherein said crocheted material of said distal and proximal sections is woven such that said proximal and distal sections unravel simultaneously.

28. The endoprosthesis according to claim 25, wherein crocheted material of said distal and proximal sections is woven from a single filament.

29. A method for implanting an endoprosthesis into a body of a patient, the endoprosthesis having a structure which is self-expandable, the structure being covered by a sheath which resists the expansion of the structure, the sheath being coupled to a release structure, said method comprising:
providing the structure covered by the sheath which retains the structure in a reduced profile;
inserting the structure which is covered by the sheath into the body of the patient such that the structure has the reduced profile during said insertion; and
actuating the release structure for removing the sheath from one or more portions of the structure to provide the self-expansion of the one or more portions to an expanded profile, the expanded profile providing for implanting the structure in the body of the patient.

30. The method of claim 29, wherein the one or more longitudinal portions are symmetric about a transverse central plane of the structure.

31. The method of claim 29, wherein one or both of the one or more longitudinal portions are non-symmetric about a transverse central plane of the structure.

32. The method according to claim 29, wherein the sheath includes a suture which is woven into a crocheted material,
the release structure being defined by a portion of the suture which extends from the sheath,
said actuation comprising displacing the release structure relative to the sheath for unraveling thereof for said removal of the sheath.

33. A method for implanting an endoprosthesis into a body of a patient, the endoprosthesis having a structure which is self-expandable, the structure being covered by a sheath which resists the expansion of the structure, the sheath being coupled to a release structure, said method comprising:
providing the structure covered by the sheath which retains the structure in a reduced profile;
actuating the release structure for removing the sheath from one or more portions of the structure to provide the self-expansion of the one or more portions to an expanded profile of the structure.
